(19) United States
(12) Patent Application Publication
(10) Pub. No.: US 2012/0143076 A1
(43) Pub. Date: Jun. 7, 2012

(54) Isolating devie, set, treatment apparatus and methods

(75) Inventors: Sebastian Wieskotten, Ober-Ramstadt (DE); Ulrich Moissl, Bad Vilbel (DE); Peter Wabel, Darmstadt (DE)

(73) Assignee: FRESENIUS MEDICAL CARE DEUTSCHLAND GMBH, Bad Homburg (DE)

(21) Appl. No.: 13/310,927

(22) Filed: Dec. 5, 2011

Related U.S. Application Data
(60) Provisional application No. 61/419,901, filed on Dec. 6, 2010.

(30) Foreign Application Priority Data
Dec. 6, 2010 (DE) 10 2010 053 413.7

(51) Int. Cl. A61B 5/053

(52) U.S. Cl. 600/547

(57) ABSTRACT

The present invention relates to an isolating device provided or intended for preventing or inhibiting a current flow between a first body section and a second body section of a patient during a bioimpedance measurement. It further relates to a set, a treatment apparatus and methods.

### CROSS REFERENCE TO RELATED APPLICATIONS

This application claims priority to U.S. Provisional Application Ser. No. 61/419,901 filed Dec. 6, 2010 and German Patent Application No. 10 2010 053 413.7 filed Dec. 6, 2010.

### FIELD OF INVENTION

The present invention relates to an isolating device. It further relates to a set, a treatment apparatus as well as methods.

### BACKGROUND OF THE INVENTION

The hydration state of a patient can be determined by means of a bioimpedance measurement. In practice, bioimpedance measurements are, e.g., performed during an extracorporeal blood treatment such as a dialysis treatment.

### SUMMARY OF THE INVENTION

One object of the present invention is to propose a device suited for being used during a bioimpedance measurement and serving for avoiding artifacts during the bioimpedance measurement. Furthermore, a set, a treatment apparatus and several methods using the device for avoiding artifacts shall be provided.

All or some of the advantages that can be obtained by means of the isolating device according to the present invention may undiminishedly also be achieved by means of the set according to the present invention and/or by means of the treatment apparatus according to the present invention and/or by means of the methods according to the present invention.

According to the present invention, an isolating device is proposed which is provided or intended for preventing or inhibiting a current flow between a first body section and a second body section of a patient during a bioimpedance measurement. The current flow to be prevented or inhibited or to be reduced is particularly an undesired current flow or a current flow disturbing or falsifying the bioimpedance measurement. The current flow to be prevented or inhibited or to be reduced may be a current flow leading to artifacts during the bioimpedance measurement. The current flow to be prevented or to be reduced may be a current (flow) that is introduced into the patient for the purpose of the bioimpedance measurement.

The set according to the present invention comprises at least one isolating device according to the present invention as well as at least one bioimpedance measuring system or apparatus.

One method according to the present invention serves for measuring the bioimpedance of a patient and comprises the use of at least one isolating device according to the present invention or of a set according to the present invention.

The treatment apparatus according to the present invention comprises at least one isolating device according to the present invention or at least one set according to the present invention. The treatment apparatus may be provided or intended for being used with an isolating device according to the present invention or with at least one set according to the present invention.

A further method according to the present invention serves for the treatment, for example, the blood treatment, of a patient. The method comprises the use of at least one isolating device according to the present invention or of a set according to the present invention or of a treatment apparatus according to the present invention.

A further method according to the present invention serves for enhancing the accuracy or the correctness or the expressiveness of resistance measurements, for example of bioimpedance measurements.

The method for enhancing the accuracy in certain embodiments comprises the application of at least one isolating device according to the present invention or of a set according to the present invention at or on the patient.

In some embodiments, the method for enhancing the accuracy comprises the provision of an isolating device according to the present invention or of a set according to the present invention, preferably without applying the set at or on the patient or connecting the set to the patient.

A further method according to the present invention serves for excluding or avoiding or reducing the occurrence of undesired current flow paths that could occur during a resistance measurement or a bioimpedance measurement following the said method through the body of a patient. This is preferably achieved by means of preliminary or preparatory, respectively, measurements, i.e. prior to the beginning of the bioimpedance measurement.

The method comprises the application of at least one isolating device according to the present invention or of a set according to the present invention at or on the patient and/or the provision of an isolating device according to the present invention or of a set according to the present invention.

In some embodiments, the method comprises the use of a treatment apparatus according to the present invention.

In certain embodiments, the methods according to the present invention have each been performed prior to the beginning of an examination and/or a treatment of a patient. In certain embodiments, the presence of the patient is not necessary. In certain embodiments, the methods according to the present invention may substantially be performed by any arbitrarily skilled or trained person.

Embodiments according to the present invention may comprise some or all of the following features in any arbitrary combination.

The term “body section” as used herein generally refers to a section or segment or portion or part of the body of a patient. Thereby, it is not decisive regarding the present invention if the patient is a human being or an animal or if the patient is healthy or ill.

The terms “first body section” and “second body section” relative to each other refer to two, in certain embodiments according to the present invention, different body sections, body parts or body areas of the patient. Thereby, the first and the second body section may each be part of the same or of two different body parts (a body part is, e.g., the arm, the torso, the leg etc.).

The first body section and the second body section do not merge in certain embodiments according to the present invention.

The first body section and the second body section comprise in some embodiments according to the present invention a shared contact surface, a shared contact area or a shared contact section. By means of the contact surface, contact area or contact section, the current flow to be prevented or inhibited takes place or may possibly take place.

In the shared contact surface, the shared contact area or section, the first body section and the second body section may touch or have contact with each other in some embodiments according to the present invention, without necessarily always touching or contacting each other herein.

A “contact” is in certain embodiments understood as a skin contact and/or a contact by means of worn clothing.

The contact may in some embodiments according to the present invention be detachable or liftable by means of movements of the patient. For example, the position of the first body section to a second body section can be changed in some embodiments according to the present invention by stretching or bending joints of the patient.

In some embodiments according to the present invention, the contact is prevented or inhibited, at least in the area of the isolating device, by arranging, plugging in or sliding in or introducing the isolating device between the first and the second body section.

For bioimpedance measurements, it may, e.g., be intended to introduce current into the first body section of the patient (the first body section may also be referred to as a measurement body section, that means a section consulted for a resistance measurement because of introducing current therein) and to measure a strength of the current or an amperage, respectively, at a third body section (the third body section being a further measurement body section; the third body section is referred to as a measurement body section as it is consulted for the measurement as well). In doing so, it is, however, not intended, to introduce current into the body or into the tissue of the patient, respectively, or to detect a current that has flowed through the body or the tissue of the patient, respectively, at a second body section (=non-measurement body section, i.e., a section into which no current is introduced or measured during the respective measurement procedure).

Due to touch or contact between, for example, the first and the second body section, the current flow may find a way through the patient's body in which, e.g., parts of the torso are not flowed through as expected. Resistances measured by means of the bioimpedance measurement may thereby significantly deviate or differ from the expected resistances.

It has to be noted that the numerical denotation of the body sections is substantially or completely irrelevant for the present invention; it substantially serves for the differentiation of the body sections from each other.

When being used accordingly, the isolating device according to the present invention may advantageously serve for preventing or inhibiting an undesired, inasmuch superficial, or direct current flow between the first body section and the second body section of the patient, which in particular can occur across a point at which the first body section and the second body section fit closely in a detachable way.

“Preventing or inhibiting a current flow” is in some embodiments according to the present invention understood as preventing or inhibiting (preferably only) a current flow between the first and the second body section, which arises or could arise by means of the contact surface or by means of the contact area between the first body section and the second body section.

In certain embodiments of the present invention, the isolating device according to the present invention is provided or intended for being fixed or attached to or at (or around) the first or to or at (or around) the second body section of the patient or is fixed or attached thereto. Additionally or alternatively, the isolating device according to the present invention may be provided or intended for being fixed or attached to or at a piece of clothing worn by the patient (in particular a piece of everyday clothes or street clothes) or is fixed or attached thereto accordingly.

In certain embodiments, the isolating device according to the present invention in at least sections thereof comprises or consists of a non-conducting material.

In some embodiments, the isolating device according to the present invention does not comprise or does not consist of a conducting material. Such a conducting material may be or may comprise an electrode.

The term “non-conducting material” as used herein, in certain embodiments refers to a material through which no current is conducted. In other embodiments, however, the material—when used—does not conduct any current during the bioimpedance measurement in orders that would have any relevant effect or influence on the resistance measurements.

Non-limiting examples for suitable non-conducting materials include cloth, plastic, leather, gum, cork, wicker, glass, card, paper, foam, ceramic, polystyrene, fur or skin and wood materials as well as combinations and/or mixtures thereof.

In certain embodiments, the isolating device is designed or embodied as an apron or a skirt, a blouse or gown or pinafore, or the like.

The isolating device according to the present invention may comprise or consist of one or more of the materials mentioned above.

The concrete design, form or shape, dimensions such as length, thickness or strength, width, cross-section, color (optionally impressed or imprinted or stamped) patterns, surface quality of any arbitrary surface, ridge, rim or edge or side such as roughness, embossing, impression or imprinting, reinforcements or stiffening, processing, preparation or manufacture, and the like, are substantially or completely insignificant for the function of the isolating device according to the present invention regarding the context of the present invention and/or the use thereof. It can be up to a person skilled in the art to select the design and/or construction required and/or desired and/or optimized for a concrete case and being the most promising one regarding the intended purpose. Any alterations, modifications, additions and/or retrenchments or reliefs, and the like that can be carried out for the isolating device according to the present invention and that do not have any significant influence or effect on the isolating function thereof are comprised by the present invention.

The concrete design or construction may be selected in dependence of the intended position of the isolating device on or at a body section or between two—adjacent and/or abutting and/or opposed and/or movable away from each other etc.—body sections or such as to be adapted to the form or shape of one or more body sections of the patient. The isolating device may be designed or embodied and/or constructed depending on the concrete case, the patient and/or the surroundings or environment (e.g., it may have a thinner construction for warmer temperatures in the examination or treatment room, or vice versa, and the like).

The isolating device may be adapted or get adapted—even on site with the patient—to the body section of the patient at or on which it is intended to be fixed or attached, wherein adaptation, e.g., as regards the quality and/or the dimension of the body section of the patient. Thus, in certain embodiments, the isolating device may be intended to be used by children as patients and may be designed or embodied accordingly; in other embodiments, the isolating device may be provided for being used by an adult. The isolating device may also specifically be designed or embodied for being applied around, e.g., thicker or thinner body parts, e.g., the patient's arms.

In certain embodiments, the isolating device according to the present invention may be designed or embodied variably or adjustably, for example by means of hook-and-loop device(s) as regards the dimensions, in particular the diameter, thereof. In this way, a flexible arrangement of the isolating device at or on the body section of the patient or at or on the patient's clothing (clothing worn by the patient) may advantageously be possible.

Without being limited thereto, an exemplary design or construction of the isolating device according to the present invention is a cuff which, in certain embodiments of the present invention, is applicable or is applied around the first or the second body section or around a part of the body section of the patient or is applied in exemplary embodiments of at least one of the methods according to the present invention.

In certain embodiments, the cuff, particularly in the state of use thereof, is designed or embodied as to be completely or in at least one section thereof closed or closable. Preferably, it is closed or closable along a cross-section continuously extending within an interior of the cuff. In some embodiments, the cross-section of the cuff is approximately, substantially or completely tube- or tubing-shaped.

In certain embodiments of the present invention, the cuff, particularly during the state of use thereof, is completely or in at least one section thereof designed or embodied as a cuff partially encompassing the first or the second body section. For example, the cuff may be designed or embodied as an arm or a leg cuff.

In certain embodiments of the present invention, the isolating device is suited and provided or intended and/or designed or embodied for being (particularly detachably) attached or fixed to or at the first or the second body section of the patient and/or at or on a piece of clothing of the patient and/or it is attached or fixed in exemplary embodiments of at least one of the methods according to the present invention.

In certain embodiments of the present invention, the isolating device is provided or intended to be fixed or attached by means of at least one fixing device or attaching device and/or it is fixed or attached in this manner. The attaching device or fixing device may be or get arranged at or on the isolating device in a firm or undetachable, respectively, or detachable manner. The kind and/or method of arranging and/or attaching or fixing the fixing device(s) at or on the isolating device or the cuff, respectively, is insignificant for the purpose of the present invention as long as the isolating action or effect of the isolating device is not substantially or only insignificantly affected (the latter could, e.g., be true when using metal buckles).

Appropriate fixing devices or attaching devices for fixing or attaching the isolating device at or on or around a body section or at or on a piece of clothing of the patient are preferably, and without being limited thereto, laces, hook-and-loop fasteners, clamping devices, ear and hook connections, tying connections, buckle connections, loop or lug connections. Thereby, combinations and/or compositions thereof and suchlike are encompassed.

In certain embodiments, the isolating device according to the present invention comprises at least one cushion device. Without being limited to any concrete design or construction, examples include gel cushions, foam cushions, gum cushions, and the like.

In some embodiments of the present invention, the isolating device according to the present invention or at least one section thereof comprise a shape or form huddling against the first and/or the second body section of the patient. In certain embodiments, the isolating device herefor comprises at least in sections thereof elements that are deformed or deformable accordingly.

In some embodiments, the isolating device according to the present invention comprises no electrode for acquiring electrical signals or electrical potential from or inducing electrical signals or electrical potential into a body section.

In some embodiments, the isolating device according to the present invention is not a blood pressure cuff.

In exemplary embodiments of at least one of the methods according to the present invention, the isolating device is arranged between the first and the second body section, for example, it is applied, inserted, slid-in or introduced.

In some embodiments of at least one of the methods according to the present invention, the isolating device is placed at or around a leg (thigh and/or lower leg), preferably up into or adjacent to the crotch.

In some embodiments of at least one of the methods according to the present invention, the isolating device is placed at or around an arm (upper arm and/or forearm), preferably up into or adjacent to the axilla.

In exemplary embodiments of at least one of the methods according to the present invention, it encompasses measuring or determining the bioimpedance.

The following and further advantages may be achieved by means of all or some embodiments of the present invention.

Thus, in certain embodiments, the present invention advantageously provides an isolating device for preventing or avoiding an undesired current flow between two body sections. This may advantageously contribute to reducing or even excluding wrong or falsified measurement results. Thus, the accuracy of a measurement method or a resistance measurement method, respectively, e.g. of a bioimpedance measurement method, may advantageously be enhanced.

Additionally, in certain embodiments, it may advantageously be possible to reduce or avoid runaway values resulting from the non-prosecution of the desired or expected (inasmuch usual) current flow path. Such runaway values or artifacts may, e.g., result from the patient's movements. Further, they may occur when the patient is wearing clothing having a large degree of freedom as regards the movement of the patient, e.g., in a case where the crotch of the patient's trousers is too low such that the thighs may touch each other and the measurement current flow could flow across a part of the thighs in parallel; or in a case in which the outerwear of the patient is broad such that the (nude) upper arm touches the (nude) upper part of the body of the patient which may also result in a falsified measurement signal. Because of establishing a contact between two body sections—between the first and the second body section or between the measurement body section and another, non-measurement body section, both reasons may lead to errors in measurement.

According to the present invention, it may advantageously be possible to avoid the calculation of an optionally wrong body composition such as the Adipose Tissue Index (ATI), the Lean Tissue Index (LTI), and the like, that may, for example, result from an undesired and optionally falsifying measurement of the one or more Cole resistances R_{E} or R_{I}.

Hence, the present invention may advantageously contribute to enhancing the accuracy of measurement of resistance measurements and/or to avoiding errors of such measurements. This may advantageously contribute to providing an improved patient safety.

### BRIEF DESCRIPTION OF THE DRAWINGS

In the following, the present invention will be described merely exemplarily with respect to the appended figures. In the figures of the drawing, identical reference numerals refer to same or identical elements. In the figures:

FIG. 1 illustrates the occurrence of runaway values during LTI or ATI measurements, respectively, and measurements of the resistance vs. time.

FIG. 2 illustrates the effect or influence of an undesired current flow or current flow path on the resistance measurement.

FIG. 3 illustrates the relationship between the LTI values and the position of the isolating device according to the present invention at or on a measurement body section of the patient.

FIG. 4 illustrates the relationship between the ATI values and the position of the isolating device according to the present invention at or on the measurement body section of the patient.

FIG. 5 shows an exemplary embodiment of an isolating device according to the present invention.

FIG. 6 shows the isolating device of FIG. 5 applied to a patient.

### DETAILED DESCRIPTION OF THE DRAWINGS

Usually, a bioimpedance measurement is performed by introducing electrical current at or in or into, respectively, a body section of the patient and detecting the intensity of the current or the amperage, respectively, at another body section of the patient, for example, by means of a hand-to-foot measurement. Both introducing and tapping or detecting or measuring is carried out by using electrodes that are brought into contact with the body, e.g., at or on one or more arms or legs. With the help of the resistance calculated, for example, the water content, the fat content, the muscle mass etc. of the patient may be concluded.

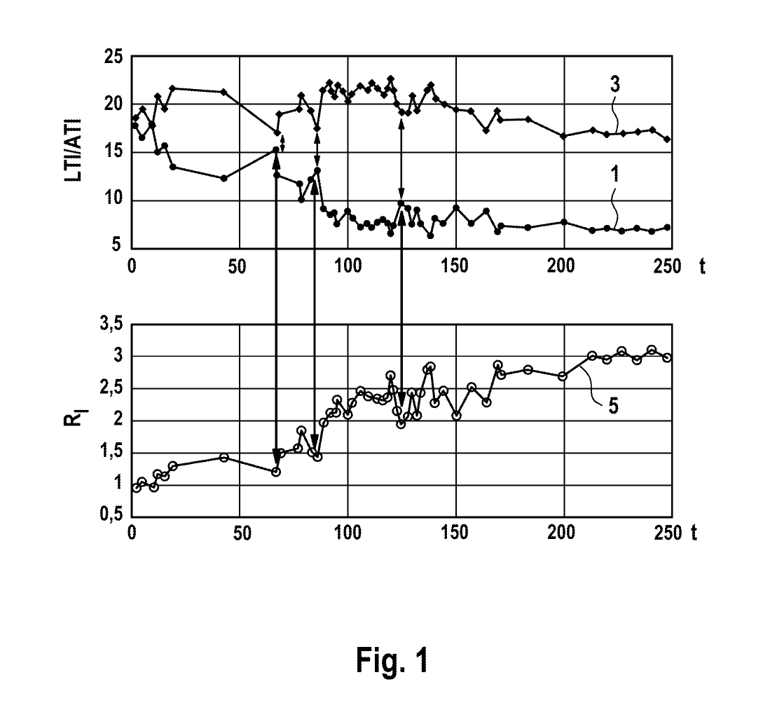

FIG. 1 illustrates the occurrence of runaway values during bioimpedance measurements of the ATI (ATI=ATM/Hgt², i.e. the Adipose Tissue Mass per square of body height or body size, respectively) and of the LTI (LTI=LTM/Hgt², i.e. the Lean Tissue Mass per square of the body height or the body size, respectively) and during measurements of the resistance verses time.

The upper graph shown in FIG. 1 shows the Lean Tissue Index (LTI) in [kg/m²] (reference numeral 1) and the Adipose Tissue Index (ATI) in [kg/m²] (reference numeral 3) verses the time t, measured in [minutes].

The lower graph shown in FIG. 1 shows the resistance R_{I} in [Ohm, Ω] (reference numeral 5) verses the time t.

The double arrows connecting the upper graph with the lower graph in FIG. 1 show runaway values of the measurements.

As can be seen from FIG. 1, the Cole resistance R_{I} significantly drops down with runaway values occurring in the LTI signal: one recognizes a drop down of the Cole resistance R_{I} with concomitant increase of the LTI and concomitant decrease of the ATI.

Reasons for such runaway values may be that the expected or usual, respectively, current flow path of the bioimpedance measurement is not prosecuted. This may be observed in case of direct skin contact between the upper arm and the torso or between the left and the right thigh if the patient is, e.g. wearing convenient or comfortable or loose, respectively, clothes as has been mentioned above.

Through a measured alteration that is only supposed to be correct in case of runaway values there is the risk of incorrectly calculating the body composition.

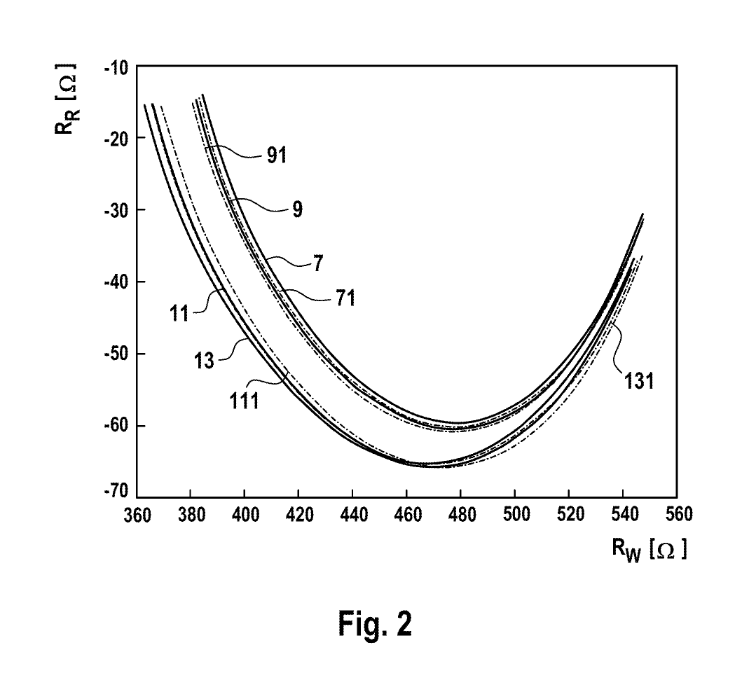

FIG. 2 illustrates the effect or influence of an undesired current flow on the measurement of the bioimpedance which is shown by means of loci of the reactance R_{R} verses the effective resistance R_{W}. In FIG. 2, the frequency fₘₑₐₛ for every locus decreases from the left to the right side or increases from the right to the left side, respectively.

Two series of measurements have been recorded at one patient or subject, respectively, wherein the patient had been examined while wearing underwear (all results have been reproduced with another patient).

In a first series of measurement, there have been measured (while using duplicate measurements):

lines having reference numerals 7 and 71: BIS measurement (bioimpedance spectroscopy), wherein the arms and the legs are splayed out (faultless measurement);
lines having reference numerals 9 and 91: BIS measurement wherein the arms are splayed out, the thighs touch each other;
lines having reference numerals 11 and 111: BIS measurement wherein the legs are splayed out, and the arm touches the torso along a distance of approximately 11 cm;
lines having reference numerals 13 and 131: BIS measurement in which the thighs and arm/the upper part of the body touch each other.

The continuous lines in FIG. 2, i.e. the lines having the reference numerals 9, 11 and 13, show the loci for measurements during which a conducting contact is established between thigh/legs or between arm and the upper part of the body, respectively. The dash-dot lines, i.e. the lines having the reference numerals 91, 111 and 131, show the results of the repeated measurements, respectively.

If the arm contacts the upper part of the body, see the lines having the reference numerals 11, 111, 13 and 131, the corresponding locus significantly deviates from the first faultless measurement (see the line having reference numeral 7 or 71, respectively).

In a second series of measurements, it has thus been examined how the locus changes when the contact between arm and the upper part of the body is reduced by means of the isolating device according to the present invention in a stepwise manner: For this series of BIS measurements, the patient's legs are always splayed out, the arm carrying the electrode always touches the upper part of the body. The isolating device according to the present invention is shifted along or across the contact area between arm and the upper part of the body in a stepwise manner into the direction of the axilla such that the length of the contact area in the direction of the upper arm is reduced from 11 cm to 0 cm.

It has been found that there is a relationship between the distance of the isolating device applied to or on a measurement body section (arm as the first body section) of the patient to a non-measurement body section (upper part of the body as the second body section) of the patient such that the resistance mainly becomes the smaller for higher measurement frequencies, the larger the contact between arm and the upper part of the body is.

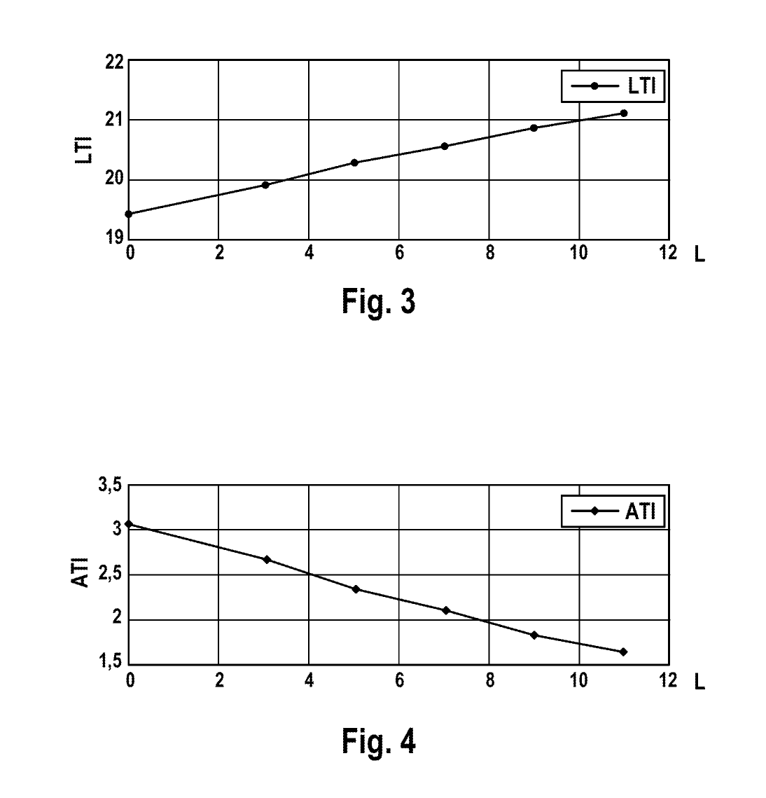

FIG. 3 illustrates the relationship between the LTI values and the position of the isolating device according to the present invention at or on the arm as a measurement body section of the patient. Thereby, the y-axis denotes the LTI in [kg/m²] and the x-axis denotes the length L of the contact area along the upper arm in [cm].

As can be seen from FIG. 3, there is a substantially linear relationship: the longer the contact (i.e. the more arm and the upper part of the body of the patient touch), the larger the LTI is.

FIG. 4 illustrates the relationship between the ATI values and the position of the isolating device according to the present invention at or on the measurement body section of the patient, wherein the y-axis denotes the ATI in [kg/m²] and the x-axis denotes the length L of the contact in [cm].

As can be seen from FIG. 4, there is also a substantially linear relationship: the longer the contact (i.e. the more arm and the upper part of the body of the patient can touch), the smaller the ATI is.

Thus, FIG. 3 and FIG. 4 impressively show the extent artifacts can take on in case of undesired current flow paths.

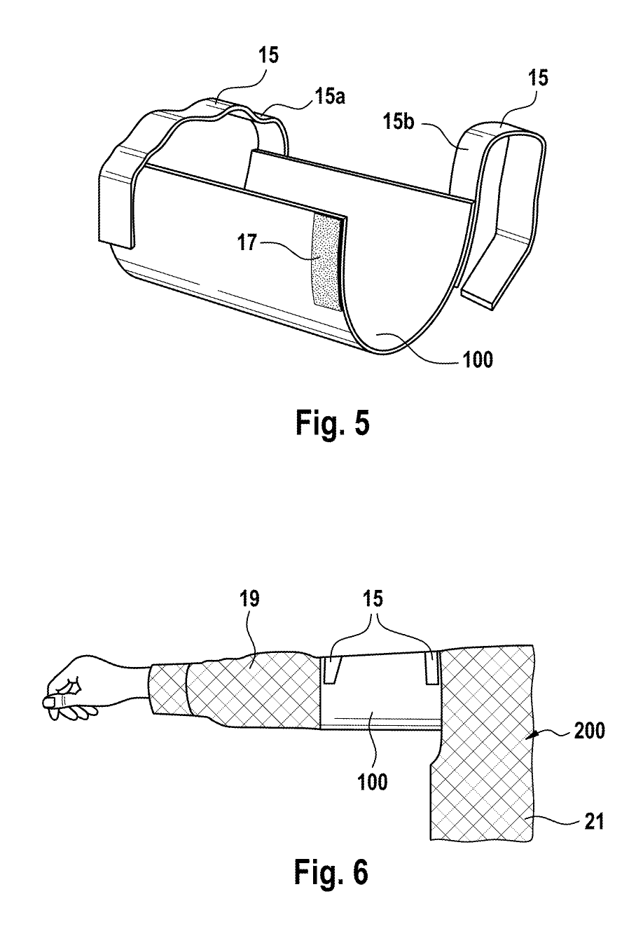

FIG. 5 shows an exemplary embodiment of an isolating device 100 according to the present invention. Here, it is a cuff present in an open state in a non-used state. Depending on the arm's circumference, the cuff has a semi-circular, i.e. partially open, cross-section up to a completely circular, i.e. closed, cross-section in a state of use.

In FIG. 5, the isolating device 100 is shown in a perspective view from the right front side slanting to the left rear side.

In the exemplary embodiment of FIG. 5, the isolating device 100 comprises two fixing devices 15.

As shown in FIG. 5, the fixing device 15a arranged at the left side is closed, i.e. it illustrates a state of fixation or attachment of the isolating device 100 to or at a body section of the patient (not shown here): The fixing device 15b arranged on—relative to the illustration of FIG. 5—right side is open which may, for example, be the case during a state of storage of the isolating device 100.

In FIG. 5, the fixing devices 15 are exemplary represented as hook-and-loop fasteners, wherein the isolating device 100 comprises corresponding complementary elements 17 provided or intended for generating a closing and/or retaining or fixing effect.

FIG. 6 shows the isolating device 100 of FIG. 5 that is arranged at a first body section 19 of a patient 200. Here, it is arranged at the right arm.

In the representation shown in FIG. 6, the isolating device 100 is arranged directly in front of the axilla of the patient 200 such that a contact between the arm and the upper part of the body, i.e. between a first body section 19 (=measurement body section), here, the arm, and a second body section 21 (=a non-measurement body section), here, the upper part of the body, is at minimum or even completely prevented.

The fixing devices 15 are closed; the isolating device 100 encompasses the patient's 200 arm.

As shown in FIG. 6, the patient 200 wears a shirt which is encompassed by the isolating device 100 as well.

What is claimed is:

1-16. (canceled)

17. An isolating device for preventing or inhibiting a current flow between a first body section and a second body section of a patient during a bioimpedance measurement.

18. The isolating device according to claim 17, wherein said device is adapted to be attached or fixed to the first body section or the second body section of the patient, or to a piece of clothing worn by the patient.

19. The isolating device according to claim 17, comprising a non-conducting material.

20. The isolating device according to claim 19, wherein the non-conducting material is selected from the group consisting of: cloth, plastic, leather, gum, cork, wicker, glass, card, paper, foam, ceramic, polystyrene, fur, skin, wood materials, and combinations and/or mixtures thereof.

21. The isolating device according to claim 17 comprising a cuff adapted to be applied around the first body section or the second body section or a part of the first or second body section of the patient.

22. The isolating device according to claim 21, wherein the cuff is adapted to be closed or closable completely or in at least one section thereof.

23. The isolating device according to claim 21, wherein the cuff is adapted to partially or completely enclose or surround the first body section or the second body section.

24. The isolating device according to claim 18, wherein the isolating device comprises at least one fixing device to attach or fix the device to the first body section, the second body section, or a piece of clothing.

25. The isolating device according to claim 24, wherein the at least one fixing device is a detachable fixing device.

26. The isolating device of claim 25, wherein the detachable fixing device is selected from the group consisting of: belts or laces, hook-and-loop fasteners, clamping devices, ear and hook connections, tying connections, buckle connections, loop or lug connections as well as combinations and/or compositions thereof.

27. The isolating device according to claim 17, comprising at least one cushion device.

28. The isolating device according to claim 17, comprising:
a form or shape huddling against the first body section, against the second body section, or both of the patient; and/or
wherein the isolating device comprises, at least in sections thereof, correspondingly deformed or deformable elements.

28. A set comprising:
at least one isolating device according to claim 17; and
at least one bioimpedance measuring system.

29. The set according to claim 28, wherein the at least one isolating device comprises two isolating devices.

30. A method for measuring the bioimpedance of a patient, comprising:
preventing or inhibiting a current flow between a first body section and a second body section of a patient; and
measuring the bioimpedance of the patient while preventing or inhibiting the current flow.

31. A treatment apparatus comprising at least one isolating device to prevent or inhibit a current flow between a first body section and a second body section of a patient during a bioimpedance measurement.

32. The treatment apparatus according to claim 31, wherein the treatment apparatus is a blood treatment apparatus for performing a hemodialysis, a hemofiltration, or a hemodiafiltration of a patient.

33. A method for the treatment of a patient, comprising the step of:
using at least one isolating device between a first body section and a second body section of a patient to prevent or inhibit a current flow during a bioimpedance measurement.

34. A method for enhancing the accuracy of resistance measurements, comprising the step of:
applying at least one isolating device between a first body section and a second body section of a patient to prevent or inhibit a current flow during a bioimpedance measurement; and/or
using a blood treatment apparatus.

35. A method for excluding, avoiding, or reducing the occurrence of undesired current flow paths through the body of a patient, which could occur during a resistance measurement or a bioimpedance measurement, comprising:
applying at least one isolating device between a first body section and a second body section of a patient to prevent or inhibit a current flow during a bioimpedance measurement; and/or
using a blood treatment apparatus.
